SEQUENCE LISTING (19 sequences)

SEQ ID NO: 1 - DNA, 723 bp, Neurospora crassa
  CDS (1)..(723)
atgaaaacctttgctactctactagcctcaattggtttggttgcagctcatgggtttgtggacaatgcgacaattggcggtcaattctaccagttctatcagccttaccaagatccctatatgggttctccacctgacaggatttccagaaagattcccggaaatggacctgttgaagatgttacgagtcttgcaatccaatgcaatgctgactcagcaccagcaaagttacatgcgagtgctgctgcgggttctacggtcactttacgttggaccatatggccagattcgcatgttggaccagtcatcacctatatggctagatgtccagatactggttgtcaagattggactccctctgccagcgataaagtctggtttaagatcaaagaaggcggtagagaaggcactagcaacgtatgggctgcaaccccgttgatgacagcaccggccaactacgaatacgccataccatcatgcttaaaaccagggtattacctggttagacacgagataattgctcttcattcggcttactcctatcctggtgcacagttctatccgggttgtcaccaattgcaagtgactggtagtgggacgaaaactccatcatctggattggtgtcttttcctggcgcctataagtccacagatcctggtgtaacatatgacgcttatcaagctgccacatacacaattcctggaccagcagtatttacctgt

SEQ ID NO: 2 - protein, 241 aa, Neurospora crassa
MKTFATLLASIGLVAAHGFVDNATIGGQFYQFYQPYQDPYMGSPPDRISRKIPGNGPVEDVTSLAIQCNADSAPAKLHASAAAGSTVTLRWTIWPDSHVGPVITYMARCPDTGCQDWTPSASDKVWFKIKEGGREGTSNVWAATPLMTAPANYEYAIPSCLKPGYYLVRHEIIALHSAYSYPGAQFYPGCHQLQVTGSGTKTPSSGLVSFPGAYKSTDPGVTYDAYQAATYTIPGPAVFTC

SEQ ID NO: 3 - DNA, 714 bp, Neurospora crassa
  CDS (1)..(714)
atgaaagtcttagcacccttggtattagcctctgcggcatctgcccatactatcttttcttccctagaagtgaatggcgtaaaccaaggtttaggtgaaggagttagagtacctacctataatggcccaattgaggatgtgacaagcgcttctattgcctgcaatggtagtccgaataccgtagcttcaacgtccaaagtgattacagttcaagctggtactaatgtcactgcaatttggcgttacatgctatcaacaactggcgattcaccagctgatgttatggacagttcgcataaagggcctacaatagcctaccttaagaaggtcgacaatgcagcaactgcatcaggcgttggtaacggttggttcaaaatccagcaagacggaatggatagctctggtgtctggggtacagaaagggttataaacggaaaagggagacacagcatcaagatacctgagtgtattgctcctggccaatacttgttgagagcggaaatgatcgcattgcatgccgctagtaactatccaggtgctcagttctacatggaatgtgctcaactgaacgtagttggtggaacaggagctaaaactcccagtacggtttcctttccaggagcgtattctggttcagatccaggggtcaagatttccatatattggccacctgttacctcgtataccgttccaggtccgtctgtgtttacgtgt

SEQ ID NO: 4 - protein, 238 aa, Neurospora crassa
MKVLAPLVLASAASAHTIFSSLEVNGVNQGLGEGVRVPTYNGPIEDVTSASIACNGSPNTVASTSKVITVQAGTNVTAIWRYMLSTTGDSPADVMDSSHKGPTIAYLKKVDNAATASGVGNGWFKIQQDGMDSSGVWGTERVINGKGRHSIKIPECIAPGQYLLRAEMIALHAASNYPGAQFYMECAQLNVVGGTGAKTPSTVSFPGAYSGSDPGVKISIYWPPVTSYTVPGPSVFTC

SEQ ID NO: 5 - protein, 14 aa, Neurospora crassa
LHASAAAGSTVTLR

SEQ ID NO: 6 - protein, 14 aa, Neurospora crassa
TPSSGLVSFPGAYK

SEQ ID NO: 7 - protein, 7 aa, Neurospora crassa
GPTIAYK

SEQ ID NO: 8 - protein, 16 aa, Neurospora crassa
IQQDGMDSSGVWGTER

SEQ ID NO: 9 - protein, 19 aa, Neurospora crassa
TPSTVSFPGAYSGSDPGVK

SEQ ID NO: 10 - DNA, 750 bp, Thermoaseus aurantiacus
atgtccttttccaagattattgcgactgctggtgttttggctagcgctagtttggtcgcaggacatggctttgttcagaacatcgtgattgatggcaagaaatactatggtggttatttggtcaatcaatatccctacatgagcaatcctcccgaagtgattgcatggtctacaactgcgactgatttaggctttgttgatggtactgggtatcaaacaccggacattatctgtcacagaggtgccaaaccaggtgctctaacagcacctgtatctccaggtggtacagttgaacttcaatggacgccttggcctgattctcatcatggcccggtcataaactacttggctccttgtaatggtgactgttcaacagtagacaaaacgcagttagagttcttcaagatagctgaatctgggttgatcaatgatgacaacccaccaggaatttgggcctcagataacttgatagcagccaataattcgtggacagttacgatcccaaccactattgcaccagggaattacgtgttaaggcacgaaattattgccctacattcagcccaaaaccaagatggtgctcaaaactatccacaatgcatcaatcttcaagtaaccggtggtggaagtgacaatcctgcaggcactttaggaaccgctttataccacgatactgatccaggtatactgattaacatctatcagaaactatcgtcctacataataccgggaccacccctgtataccgga

SEQ ID NO: 11 - DNA, 30 bp, Artificial Sequence
  Synthetic DNA
aagcgcggcggtggctttgtggacaatgcg

SEQ ID NO: 12 - DNA, 30 bp, Artificial Sequence
  Synthetic DNA
caagaaagctgggtattaacaggtaaatac

SEQ ID NO: 13 - DNA, 30 bp, Artificial Sequence
  Synthetic DNA
aagcgcggcggtggccatactatcttttct

SEQ ID NO: 14 - DNA, 30 bp, Artificial Sequence
  Synthetic DNA
caagaaagctgggtattaacacgtaaacac

SEQ ID NO: 15 - DNA, 30 bp, Artificial Sequence
  Synthetic DNA
aagcgcggcggtggctttgttcagaacatc

SEQ ID NO: 16 - DNA, 30 bp, Artificial Sequence
  Synthetic DNA
caagaaagctgggtattatccggtatacag

SEQ ID NO: 17 - DNA, 46 bp, Artificial Sequence
  Synthetic DNA
ggggacaagtttgtacaaaaaagcaggctcaaagcgcggcggtggc

SEQ ID NO: 18 - DNA, 30 bp, Artificial Sequence
  Synthetic DNA
ggggaccactttgtacaagaaagctgggta

SEQ ID NO: 19 - protein, 5 aa, Artificial Sequence
  Synthetic peptide
KRGGG

(19) United States
(12) Patent Application Publication
(10) Pub. No.: US 2013/0217076 A1
(43) Pub. Date: Aug. 22, 2013

(54) POLYPEPTIDE CAPABLE OF ENHANCING CELLULOSIC BIOMASS DEGRADATION

(71) Applicants: Noriko SHISA, Nisshin-shi (JP); Nobuhiro ISHIDA, Seto-shi (JP); Chie IMAMURA, Nagoya-shi (JP); Shigeharu MORIYA, Kawasaki-shi (JP); Jun KIKUCHI, Yokohama-shi (JP)

(72) Inventors: Noriko SHISA, Nisshin-shi (JP); Nobuhiro ISHIDA, Seto-shi (JP); Chie IMAMURA, Nagoya-shi (JP); Shigeharu MORIYA, Kawasaki-shi (JP); Jun KIKUCHI, Yokohama-shi (JP)

(21) Appl. No.: 13/768,841

(22) Filed: Feb. 15, 2013

(30) Foreign Application Priority Data
Feb. 17, 2012 (JP) 2012-033307

(51) Int. Cl. C07K 14/37

(52) U.S. Cl. CPC C07K 14/37
USPC 435/99; 530/371; 536/23.74; 435/320.1; 435/165

(57) ABSTRACT

The saccharification efficiency of cellulase is enhanced within reaction temperature regions of general fermenting microorganisms other than heat-resistant yeast. Cellulosic biomass is saccharified with cellulase in the presence of a polypeptide comprising the amino acid sequence shown in SEQ ID NO: 2 or 4 and having a function of enhancing the saccharifying activity of cellulase.

### CROSS REFERENCE TO RELATED APPLICATIONS

This application claims priority based on Japanese Patent Application No. 2012-033307 filed Feb. 17, 2012, the contents of all of which are incorporated herein by reference in their entirety.

### BACKGROUND OF THE INVENTION

1. Field of the Invention

The present invention relates to polypeptides enhancing the enzyme activity of enzymes saccharifying cellulosic biomass, nucleic acids encoding the polypeptides, and a method for producing ethanol using the polypeptides.

2. Background Art

Cellulosic biomass is effectively used as a raw material for useful alcohol such as ethanol or organic acid. Cellulosic biomass is mainly composed of cellulose, hemicellulose, and lignin. When cellulosic biomass is used as a raw material for alcohol or organic acid, cellulose or hemicellulose should be efficiently saccharified. Known methods for saccharifying cellulosic biomass are methods using concentrated sulfuric acid or dilute sulfuric acid, and methods using enzymes such as cellulase and hemicellulose.

Saccharification methods that use enzymes have various advantages compared with methods that use concentrated sulfuric acid or dilute sulfuric acid, but they require the use of large quantities of expensive enzymes in order to achieve sufficient saccharification efficiency. Specifically, when alcohol or organic acid is produced from cellulosic biomass by a saccharification method using an enzyme, increased cost due to the increased amount of the enzyme used poses a significant problem.

As a technique for reducing the amount of an enzyme to be used, for example, as described in JP 2007-523646A, a technique of using a specific protein is known. More specifically, JP 2007-523646A discloses a protein capable of enhancing the cellulose-saccharifying activity of cellulase, which is a protein isolated from a fungus having heat resistance. In addition, the heat-resistant fungus disclosed in JP 2007-523646A is Thermoascus aurantiacus. However, the protein disclosed in JP 2007-523646A capable of enhancing the cellulose-saccharifying activity of cellulase is derived from a heat-resistant fungus, and thus it has an optimum temperature within relatively a high temperature region. Therefore, there is a constraint in the case of so-called simultaneous saccharification and fermentation (system by which saccharification by cellulase and alcohol fermentation are performed simultaneously), such that heat-resistant yeast must be used.

### SUMMARY OF THE INVENTION

As described above, within the reaction temperature regions of general fermenting microorganisms other than microorganisms such as heat-resistant yeast having its optimum temperature region within a high temperature region, there is no known substance having a function of enhancing the saccharification efficiency of cellulase. Therefore, an object of the present invention is to provide polypeptides having a function of enhancing the saccharification efficiency of cellulase within reaction temperature regions of general fermenting microorganisms other than heat-resistant yeast, nucleic acids encoding the polypeptides, and a method for producing ethanol using the polypeptides.

As a result of intensive studies to achieve the above object, the present inventors have succeeded in identifying polypeptides having a function of enhancing the saccharifying activity of cellulase from among Neurospora crassa-derived crystalline cellulose binding proteins, and thus have completed the present invention.

The present invention encompasses the following (1) to (11).
(1) An isolated polypeptide according to any one of the following (a) to (c):
(a) a polypeptide comprising the amino acid sequence shown in SEQ ID NO: 2 or 4;
(b) a polypeptide having 70% or more identity with the amino acid sequence shown in SEQ ID NO: 2 or 4 and having a function of enhancing the saccharifying activity of cellulase; and
(c) a polypeptide having an amino acid sequence that has a substitution, a deletion, an addition, or an insertion of one or a plurality of amino acid residues with respect to the amino acid sequence shown in SEQ ID NO: 2 or 4, and having a function of enhancing the saccharifying activity of cellulase.
(2) The polypeptide according to (1), which is capable of binding to crystalline cellulose.
(3) The polypeptide according to (1), which is derived from Neurospora crassa.
(4) A gene encoding the polypeptide of any one of (1) to (3) above.
(5) An expression vector containing the gene of (4) above.
(6) A saccharification method, comprising a step of saccharifying cellulosic biomass with cellulase in the presence of the polypeptide of any one of (1) to (3) above.
(7) A method for producing alcohol, comprising a step of saccharifying cellulosic biomass with cellulase in the presence of the polypeptide of any one of (1) to (3) above, and performing alcohol fermentation using a sugar component as a raw material.
(8) The method for producing alcohol according to (7), wherein the alcohol fermentation is performed using a recombinant microorganism transformed with a gene encoding the polypeptide of any one of (1) to (3) above.
(9) The method for producing alcohol according to (8), wherein the recombinant microorganism is recombinant yeast.
(10) The method for producing alcohol according to (7), wherein the alcohol fermentation is performed by a microorganism having an optimum reaction temperature region of 45° C. or less.
(11) A composition for enhancing cellulase activity comprising the polypeptide of any one of (1) to (3) above.

### EFFECT OF THE INVENTION

According to the present invention, the saccharifying activity of cellulase can be enhanced within a temperature region lower than the optimum temperature region of heat-resistant yeast. Specifically, according to the present invention, the rate of saccharifying cellulosic biomass can be improved by enhancing the saccharifying activity of cellulase. Also, according to the present invention, enhancement of the saccharifying activity of cellulase can improve the rate of saccharifying cellulosic biomass and the yield of ethanol from the cellulosic biomass.

### BRIEF DESCRIPTION OF THE DRAWINGS

FIG. 1 is a photograph showing the result of SDS polyacrylamide electrophoresis for a crystalline cellulose binding protein.

FIG. 2 shows the amino acid sequence of Neurospora crassa-derived GH61 (TF1) (SEQ ID NO: 2).

FIG. 3 shows the amino acid sequence of Neurospora crassa-derived GH61 (TF2) (SEQ ID NO: 4).

FIG. 4 is a characteristic diagram showing the results of an evaluation test for evaluating biomass degradation when steamed napier grass was used as biomass.

FIG. 5 is a characteristic diagram showing the results of an evaluation test for evaluating biomass degradation when Castanopsis sieboldii, cedar, and napier grass were used as biomass.

FIG. 6 is a characteristic diagram showing the result of an evaluation test for evaluating biomass degradation at each reaction temperature when each partially purified crystalline cellulose binding protein was added externally.

FIG. 7 is a characteristic diagram showing the results of a fermentation test when each partially purified crystalline cellulose binding protein was not added externally.

FIG. 8 is a characteristic diagram showing the results of a fermentation test when each partially purified crystalline cellulose binding protein was added externally.

### DESCRIPTION OF THE PREFERRED EMBODIMENTS

The present invention will be described in detail as follows.

### Polypeptide According to the Present Invention

The polypeptide according to the present invention is a crystalline cellulose binding protein or a polypeptide found to have a novel function of enhancing the saccharifying activity of cellulase. A specific example of the polypeptide according to the present invention is a Neurospora crassa-derived polypeptide. Among examples of the polypeptide according to the present invention, amino acid sequences of the Neurospora crassa-derived polypeptide are shown in SEQ ID NO: 2 and 4, respectively. Furthermore, the nucleotide sequences of genes encoding the polypeptides consisting of the amino acid sequences shown in SEQ ID NO: 2 and 4 are shown in SEQ ID NO: 1 and 3. Here, the polypeptide according to the present invention is not limited to the Neurospora crassa-derived polypeptides consisting of the amino acid sequences shown in SEQ ID NO: 2 and 4, and may be a polypeptide from any organism. Also, the polypeptide according to the present invention may be a polypeptide from a fungus such as yeast, a bacterium, an animal, a plant, an insect, or an algae.

More specifically, the polypeptide according to the present invention is not limited to the polypeptide consisting of the amino acid sequence shown in SEQ ID NO: 2 or 4, and may be a gene that is in a paralog relationship or in a homolog relationship in a narrow sense with the polypeptide of the present invention, even if the amino acid sequence encoded by such gene differs from that of the polypeptide.

Furthermore, the polypeptide according to the present invention may be a polypeptide that has an amino acid sequence having 70% or more, preferably 80% or more, more preferably 90% or more, and most preferably 95% or more sequence identity with the amino acid sequence shown in SEQ ID NO: 2 or 4, and has a function of enhancing the saccharifying activity of cellulase. A sequence identity value can be calculated using a BLASTN or BLASTX program equipped with the BLAST algorithm (default setting). In addition, a sequence identity value is found by calculating the number of amino acid residues that completely match upon pairwise alignment analysis for a pair of amino acid sequences, and then calculating the proportion of the number of the above amino acid residues in the total number of amino acid residues compared.

Furthermore, the polypeptide according to the present invention may be a polypeptide having an amino acid sequence that has a substitution, a deletion, an insertion, or an addition of one or a plurality of amino acids with respect to the amino acid sequence shown in SEQ ID NO: 2 or 4, and having a function of enhancing the saccharifying activity of cellulase. Here, the term “a plurality of (amino acids)” refers to 2 to 30, preferably 2 to 20, more preferably 2 to 10, and most preferably 2 to 5 amino acids, for example.

Furthermore, the polypeptide according to the present invention may be a polypeptide that is encoded by a gene hybridizing under stringent conditions to all or a part of the complementary strand of a nucleic acid consisting of the nucleotide sequence of SEQ ID NO: 1 or 3 and has a function of enhancing the saccharifying activity of cellulase. The term “stringent conditions” as used herein refers to conditions under which, namely, a specific hybrid is formed but a non-specific hybrid is not formed. Such stringent conditions can be adequately determined by referring to, Molecular Cloning: A Laboratory Manual (Third Edition), for example. Specifically, stringency can be set on the basis of the temperature for Southern hybridization and the concentration of a salt contained in the solution, and the temperature for a washing step of Southern hybridization and the concentration of a salt contained in the solution. More specific examples of stringent conditions include a sodium concentration ranging from 25 mM to 500 mM, and preferably ranging from 25 mM to 300 mM, and a temperature ranging from 42° C. to 68° C., and preferably ranging from 42° C. to 65° C. Even more specific examples of stringent conditions include 5×SSC (83 mM NaCl, 83 mM sodium citrate) and a temperature of 42° C.

Whether or not the above-mentioned polypeptide having an amino acid sequence differing from the amino acid sequence shown in SEQ ID NO: 2 or 4 has a function of enhancing the saccharifying activity of cellulase can be verified as follows. First, the polypeptide is isolated according to a conventional method. Next, a reaction solution containing cellulosic biomass from which soluble sugar has been removed, the polypeptide, and commercially available cellulase is prepared, and then a reaction of saccharifying with cellulase is performed under conditions of 45° C. and about 16 hours, for example. Also, for comparison, a reaction solution having a composition similar to that of the above reaction solution except for not containing the polypeptide is prepared, and then a reaction of saccharifying with cellulase is performed under the same conditions. After completion of the reaction, soluble sugar contained in each reaction solution is detected and then the soluble sugar contents are compared. When the soluble sugar content in the reaction solution containing the polypeptide is significantly higher than the soluble sugar content in the reaction solution containing no polypeptide, it can be concluded that the polypeptide has a function of enhancing the saccharifying activity of cellulase. In addition, a technique for measuring soluble sugar contained in a reaction solution is not particularly limited. An example thereof is a method that involves adding tetrazolium blue to a reaction solution, boiling the solution for about 10 minutes, measuring absorbance at 660 nm, and thus quantitatively determining reduced ends of soluble sugar.

The polypeptide according to the present invention can exhibit a function of enhancing the saccharification efficiency of cellulase even within a high temperature region, such as the growth temperature region of heat-resistant yeast, and a temperature region lower than a high temperature region, such as the growth temperature region of heat-resistant yeast.

### Cellulase

Here, the term “cellulase” refers to a generic name for enzymes having activity of hydrolyzing glycosidic bonds of cellulose. Examples of enzymes composing cellulase include, exo-type cellobiohydrolase (CBH1 and CBH2), which liberates cellobiose from an end of crystalline cellulose, endo-type endoglucanase (EG), which is unable to degrade crystalline cellulose but is able to randomly cleave non-crystalline cellulose (amorphous cellulose) chain, and β glucosidase, which catalyzes a hydrolysis reaction of β-glycosidic bonds.

In addition, as cellulase, conventionally known cellulase can be adequately used. Also, cellulase may be chemically synthesized cellulase or a purified microbial product. Also, as cellulase, a commercially available cellulase preparation can be used. Also, the polypeptide according to the present invention can enhance the saccharifying activity of a microorganism through co-existence with the microorganism expressing cellulose; that is, a microorganism capable of hydrolyzing cellulosic biomass. An example of a microorganism highly capable of secreting and producing cellulase is Trichoderma reesei. Specifically, the polypeptide according to the present invention can enhance the saccharifying activity of Trichoderma reesei. Examples of such microorganisms capable of generating cellulase include Aspergillus niger, A. foetidus, Alternaria alternata, Chaetomium thermophile, C. globosus, Fusarium solani, Irpex lacteus, Neurospora crassa, Cellulomonas fimi, C. uda, Erwinia chrysanthemi, Pseudomonas fluorescence, and Streptmyces flavogriseus.

### Saccharification

The term “cellulosic biomass (to be subjected to saccharification)” refers to biomass containing the crystal structure of cellulose fiber and a complex of hemicellulose and lignin. In particular, the crystal structure of cellulose fiber and hemicellulose are handled as polysaccharides contained in cellulosic biomass. Examples of cellulosic biomass include waste such as lumber from thinning, construction and demolition waste, industrial waste, domestic waste, agricultural waste, waste lumber, forest residues, and waste paper. Examples of cellulosic biomass further include corrugated cardboard, waste paper, old newspaper, magazine, pulp, and pulp sludge. Further examples of cellulosic biomass include waste lumber such as sawdust and wood shavings, and pellets produced by pulverizing, compressing, and then shaping forest residues, waste paper, or the like.

Cellulosic biomass may be used in any form; however, so-called soft biomass is preferably compressed in advance and so-called hard biomass is preferably pulverized in advance. The term “compression of soft biomass” refers to relaxing/disrupting biomass tissue by applying predetermined pressure to soft biomass. For compression, a compressor that is generally used in the filed of foods, agriculture, or the like can be used. Also, the term “pulverization of hard biomass” refers to pulverizing biomass using an apparatus such as a cutter mill. For pulverization, hard biomass is preferably partially pulverized to about a mean diameter, ranging from 0.1 mm to 2 mm, for example.

Saccharification is treatment that causes cellulase and/or a microorganism capable of secreting and producing cellulase to act on the above-mentioned cellulosic biomass. Through saccharification, cellulose and hemicellulose contained in cellulosic biomass are saccharified to result in monosaccharide (soluble sugar) such as glucose, mannose, galactose, xylose, or arabinose.

The above-mentioned polypeptide according to the present invention can enhance the saccharifying activity of cellulase through saccharification, so that it can improve the amount of soluble sugar generated with respect to the amount of cellulosic biomass that has been introduced. In other words, the above-mentioned polypeptide according to the present invention is caused to be present in a reaction system for saccharification, so that cellulosic biomass can be efficiently saccharified and the production amount of target soluble sugar can be improved.

### Alcohol Fermentation

The term “alcohol fermentation using the polypeptide according to the present invention” refers to biosynthesis of alcohol from sugar obtained by saccharification of cellulosic biomass by cellulase. In particular, with the use of the polypeptide according to the present invention, cellulosic biomass can be efficiently saccharified as described above, and thus the production amount of cellulosic biomass-derived sugar can be improved. Therefore, alcohol yield resulting from alcohol fermentation can also be improved by the use of the polypeptide according to the present invention.

In particular, alcohol fermentation using the polypeptide according to the present invention is preferably so-called simultaneous saccharification and fermentation. The term “simultaneous saccharification and fermentation” refers to a situation in which a step of saccharifying cellulosic biomass by cellulase and a step of ethanol fermentation using glucose generated by saccharification as a sugar source proceed simultaneously. Here, conventionally known yeast capable of performing alcohol fermentation can be used for alcohol fermentation.

Examples of such yeast include, but are not particularly limited to, yeast strains such as Candida shehatae, Pichia stipitis, Pachysolen tannophilus, Saccharomyces cerevisiae, and Schizosaccaromyces pombe. In particular, Saccharomyces cerevisiae is preferred. Also, yeast to be used herein may be an experimental strain to be used for convenience of experiments or an industrial strain (practical strain) to be used for practical usefulness. Examples of industrial strains include yeast strains to be used for producing wine, refined sake, or distilled spirits. Also, yeast capable of performing alcohol fermentation may be wild-type yeast, mutant yeast prepared by introducing a mutation into wild-type yeast, or recombinant yeast modified by introduction or deletion of predetermined gene(s).

In particular, recombinant yeast prepared by introducing a gene encoding the above polypeptide according to the present invention so that the gene can be expressed is preferably used. Specifically, for example, recombinant yeast can be prepared by introducing the gene consisting of the nucleotide sequence shown in SEQ ID NO: 1 or 3 so that the gene can be expressed within the yeast. Examples of a promoter that can be used for a gene to be introduced include, but are not particularly limited to, a glyceraldehyde-3-phosphate dehydrogenase gene (TDH3) promoter, a 3-phosphoglyceratekinase gene (PGK1) promoter, and a high osmolarity response 7 gene (HOR7) promoter. A pyruvate decarboxylase gene (PDC1) promoter is particularly preferred because of its high capacity to enable high-level expression of a target downstream gene. In addition to these examples, a TEF1 gene promoter, an ADH1 gene promoter, a TPI1 gene promoter, a HXT7 gene promoter, and a PYK1 gene promoter can be used.

Specifically, the above genes may be introduced into the yeast genome, together with a promoter for controlling expression and other expression control regions.

Also, as a method for introducing the above genes, any technique conventionally known as a method for yeast transformation can be applied. Specifically, the above genes can be introduced by electroporation described in “Meth. Enzym., 194, p182 (1990),” an spheroplast method described in “Proc. Natl. Acad. Sci. U.S.A., 75 p. 1929 (1978),” a lithium acetate method described in “J. Bacteriology, 153, p. 163 (1983), Proc. Natl. Acad. Sci. U.S.A., 75 p. 1929 (1978),” and methods described in “Methods in yeast genetics, 2000 Edition: A Cold Spring Harbor Laboratory Course Manual,” and the like, for example. However, the examples thereof are not limited to these methods.

When alcohol fermentation using the polypeptide according to the present invention is performed by simultaneous saccharification and fermentation, the above cellulase, the polypeptide according to the present invention, and the above yeast (which may be recombinant yeast) are added to a medium containing cellulosic biomass (which may be pre-treated), and then the recombinant yeast is cultured within a predetermined temperature range. Culture temperatures can be set to, but are not particularly limited to, range from 25° C. to 45° C. in view of ethanol fermentation efficiency, and preferably to range from 30° C. to 40° C. In particular, the polypeptide according to the present invention can enhance the saccharifying activity of cellulase within the above temperature ranges. In other words, the optimum temperature range within which the polypeptide according to the present invention enhances the saccharifying activity of cellulase almost completely corresponds to a temperature range within which the above general yeast can perform alcohol fermentation. Therefore, when alcohol fermentation is performed using the polypeptide according to the present invention while enhancing the saccharifying activity of cellulase, there is no need to use any heat-resistant yeast, and yeast capable of performing alcohol fermentation can be widely used.

Also, upon alcohol fermentation, the pH of a culture solution is not particularly limited. For example, the pH of a culture solution is preferably set to range from 4 to 6. Also, upon alcohol fermentation, a reaction solution may be stirred or shaken.

A method for alcohol production using the present invention involves recovering alcohol from media after alcohol fermentation. A method for alcohol recovery is not particularly limited, and any conventionally known method can be applied. For example, after the above alcohol fermentation has been completed, a liquid layer containing alcohol is separated from a solid layer containing yeast and solid components by solid-liquid separation procedures. Subsequently, alcohol contained in the liquid layer is separated/purified by a distillation method, so that highly purified alcohol can be recovered. In addition, the purification degree of alcohol can be adequately adjusted depending on the purpose of use of alcohol.

### EXAMPLES

Hereafter, the present invention is described in greater detail with reference to the examples, although the technical scope of the present invention is not limited to the following examples.

### Example 1

### Purification of Crystalline Cellulose Binding Protein

Obtainment of a protein binding to crystalline cellulose from a culture supernatant solution of a filamentous bacterium (Neurospora crassa NBRC 6067) was attempted.

First, the filamentous bacterium Neurospora crassa (NBRC 6067) was cultured by the following method. The filamentous bacterium Neurospora crassa (NBRC 6067) was inoculated into 100 ml of a DPY medium (CMC (1 g), glucose (1 g), polypeptone (1 g), yeast extract (0.5 g), KH₂PO₄ (0.5 g), and MgSO₄.7H₂O (0.05 g) dissolved in distilled water to 100 ml) supplemented with carboxy methyl cellulose (CMC, SIGMA-ALDRICH) as a carbon source (hereinafter, DPY+CMC medium), followed by 4 days of shake culture at 30° C. and 120 rpm for 4 days.

A crystalline cellulose binding protein was prepared from the culture supernatant solution. 4 g of crystalline cellulose (Avicel PH-101, Sigma-Aldrich) was added to 50 ml of the above obtained culture supernatant solution and then the solution was stirred with a stirrer for 5 minutes. After precipitation of Avicel, the supernatant was removed with a pipette. The resultant was washed twice with 20 ml of wash buffer (1 M (NH₄)₂SO₄, 0.1 M Tris-HCl (pH7.0)) and then a syringe was filled with crystalline cellulose. 30 ml of sterile water or 20 ml of 50 mM Tris NaOH (pH 12.5) was used for elution.

The collected solution was concentrated with an ultrafiltration membrane (NANOSEP 10K OMEGA, PALL) and then SDS-PAGE was performed using a 14% SDS polyacrylamide gel (TEFCO). Specific experimental procedures for SDS-PAGE were performed according to Molecular Cloning: A Laboratory Manual (Cold Spring Harbor Laboratory).

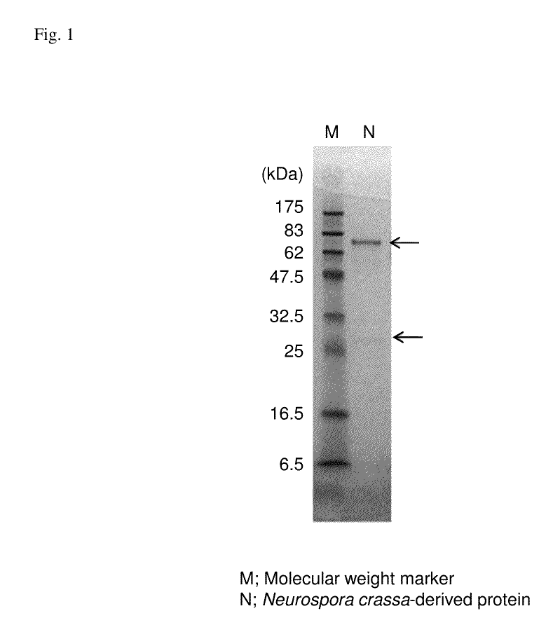

FIG. 1 shows the result of SDS polyacrylamide electrophoresis. As shown in FIG. 1, two fragments corresponding to about 70 kDa and about 30 kDa were confirmed. It was confirmed based on the result that a protein binding to crystalline cellulose was present in the culture supernatant solution of the filamentous bacterium Neurospora crassa. As a result of a survey of database, a fragment of about 70 kDa was inferred to be cellobiohydrolase possessing a cellulose binding domain. A fragment of about 30 kDa remained unidentified. As described in the following Examples, identification of this fragment was attempted.

### Example 2

### Identification of Crystalline Cellulose Binding Protein

Identification of the peptide sequence of the filamentous bacterium Neurospora crassa (NBRC 6067)-derived crystalline cellulose binding protein confirmed in Example 1 by LC-MS/MS analysis and identification of the protein through comparison with an existing database were attempted.

The fragment of about 30 kDa (FIG. 1) confirmed by SDS-PAGE in Example 1 was excised from the gel and then collected in an eppen tube. The fragment was dissolved and then treated with trypsin (Promega). The thus prepared sample was subjected to LC-MS/MS analysis.

The prepared sample was measured with a mass spectrometer while separating/concentrating the sample by reverse phase chromatography. The thus obtained results were compared with the existing database. Subsequently, the peptide fragment was randomly disrupted at the positions of peptide linkage using an argon gas, the masses of the degradation products were compared with the existing database, and thus the sequences of the peptide fragments obtained by trypsin treatment were identified.

As a result of LC-MS/MS analysis, 5 types of peptide sequence were identified successfully. The thus identified peptide sequences are as follows.

(SEQ ID NO: 5)
“Leu-His-Ala-Ser-Ala-Ala-Ala-Gly-Ser-Thr-Val-Thr-
Leu-Arg”
(SEQ ID NO: 6)
“Thr-Pro-Ser-Ser-Gly-Leu-Val-Ser-Phe-Pro-Gly-Ala-
Tyr-Lys”
(SEQ ID NO: 7)
“Gly-Pro-Thr-Ile-Ala-Tyr-Lys”
(SEQ ID NO: 8)
“Ile-Gln-Gln-Asp-Gly-Met-Asp-Ser-Ser-Gly-Val-Trp-
Gly-Thr-Glu-Arg”
(SEQ ID NO: 9)
“Thr-Pro-Ser-Thr-Val-Ser-Phe-Pro-Gly-Ala-Tyr-Ser-
Gly-Ser-Asp-Pro-Gly-Val-Lys”

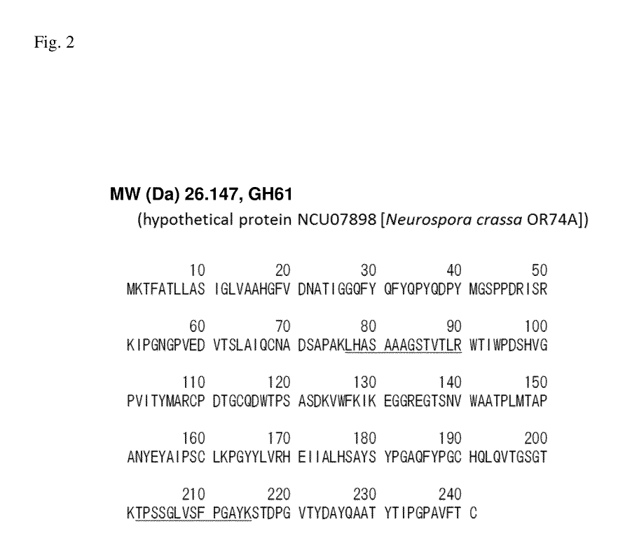

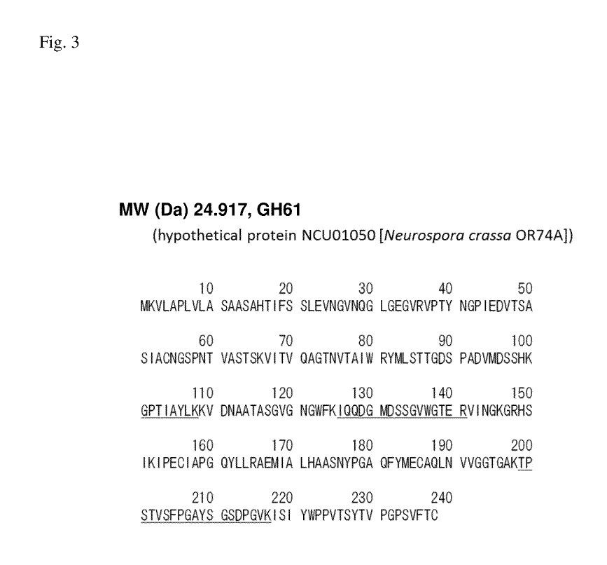

The existing database was searched for the thus identified peptide sequences to find proteins having the sequences. As a result, it was confirmed that peptides having the same sequences as the above-identified peptide sequences were present in the proteins of Neurospora crassa NCU07898 and NCU01050. The amino acid sequences of the two thus identified types of protein are shown in FIG. 2 and FIG. 3, respectively. In FIG. 2 and FIG. 3, the above 5 amino acid sequences identified by LC-MS/MS analysis are underlined.

Neurospora crassa NCU07898 is referred to as Neurospora crassa-derived GH61 (TF1) or simply TF1. Similarly, NCU01050 is referred to as Neurospora crassa-derived GH61 (TF2) or simply TF2.

### Example 3

### Synthesis of Gene Encoding Crystalline Cellulose Binding Protein and Expression Thereof in Yeast

Artificially synthesized genes optimized for expression in Saccharomyces cerevisiae were designed and synthesized (Operon) from the amino acid sequences of Neurospora crassa-derived GH61 (TF1) and (TF2) (GeneBank accession nos. EAA33178.1 and EAA32426.1, respectively) and Thermoaseus aurantiacus-derived GH61 (GeneBank accession no. ABW56451.1). The artificially synthesized gene from Neurospora crassa-derived GH61 (TF1) is shown in SEQ ID NO: 1. The artificially synthesized gene from Neurospora crassa-derived GH61 (TF2) is shown in SEQ ID NO: 3. The artificially synthesized gene from Thermoaseus aurantiacus-derived GH61 is shown in SEQ ID NO: 10. In addition, hereinafter, Thermoaseus aurantiacus-derived GH61 may also be simply referred to as “TA.”

On the basis of the thus designed nucleotide sequence information, a region encoding a signal sequence was predicted using SignalP (Jannick Dyrlov Bendtsen, Henrik Nielsen, Gunnar von Heijne and Soren Brunak. Improved prediction of signal peptides: SignalP 3.0. J. Mol. Biol., 340: 783-795, 2004). A pair of primers for amplification of a region encoding a mature protein from which the predicted signal sequence had been removed was designed as follows.

### A Pair of Primers for Removal of Signal Sequence in TF1

TF1-F:
(SEQ ID NO: 11)
5′-AAGCGCGGCGGTGGCTTTGTGGACAATGCG
TF1-R:
(SEQ ID NO: 12)
5′-CAAGAAAGCTGGGTATTAACAGGTAAATAC

### A Pair of Primers for Removal of Signal Sequence in TF2

TF2-F:
(SEQ ID NO: 13)
5′-AAGCGCGGCGGTGGCCATACTATCTTTTCT
TF2-R:
(SEQ ID NO: 14)
5′-CAAGAAAGCTGGGTATTAACACGTAAACAC

### A Pair of Primers for Removal of Signal Sequence in TA

TA-F:
(SEQ ID NO: 15)
5′-AAGCGCGGCGGTGGCTTTGTTCAGAACATC
TA-R:
(SEQ ID NO: 16)
5′-CAAGAAAGCTGGGTATTATCCGGTATACAG

Also, a protease cleavage sequence (KRGGG; SEQ ID NO: 19) was added to the N-terminus of the above mature protein and a pair of primers for adding the attB site and the attP site was designed for introduction into a vector.

Stan-GW-F:
(SEQ ID NO: 17)
5′-GGGGACAAGTTTGTACAAAAAAGCAGGCTCAAAGCGCGGCGGTG
GC
Stan-GW-R:
(SEQ ID NO: 18)
5′-GGGGACCACTTTGTACAAGAAAGCTGGGTA

An insert fragment having the attB site and the attP site to be introduced into a vector was obtained by PCR using as a template an artificially synthesized gene synthesized using these primers. The thus obtained insert fragment was incorporated into a pDONR207 vector (Invitrogen) using Gateway BP clonase (Invitrogen), so that entry clones were prepared (pTF1-ENT, pTF2-ENT, and pTA-ENT).

A destination vector (pESC-HIS-MO2-GW vector) was constructed using a Gateway vector conversion system (Invitrogen) on the basis of a pESC-HIS-MO2 vector (Toyota Central R&D Labs., Inc.); that is, a S. cerevisiae-E. coli shuttle vector. An entry clone and the destination vector were reacted using Gateway LR clonase (Invitrogen), so as to construct an expression vector (pESC-TF1-HIS, pESC-TF2-HIS, or pESC-TA-HIS).

The S. cerevisiae YPH499 strain (Stratagene, MATa, ura3-52, lys2-801, ade2-101, trp1Δ63, his3Δ200, leu2Δ1) was transformed according to the method of Amberg et al. (Amberg D C, Burke D J, Strathern J N. Methods in Yeast Genetics: a Cold Spring Harbor Laboratory Course Manual. Cold Spring Harbor, N.Y.: Cold Spring Harbor Laboratory Press; 2005). Proteins were expressed by the recombinant yeast as follows. The strain was inoculated into 40 ml of YPD medium (yeast extract (10 g/L), peptone (20 g/L), D-glucose 20 g/L)), followed by 4 days of shake culture at 30° C. The thus obtained culture supernatant was concentrated using a ultrafilter membrane with a pore size of 30 kDa and an ultrafilter membrane with a pore size of 10 kDa, and then the resultant was washed 3 times with 50 mM sodium acetate buffer (pH 5.0), so that solvent exchange was performed.

### Example 4

### Evaluation of Biomass Degradation by Crystalline Cellulose Binding Protein

The above obtained protein preparation was adjusted to have a concentration of 0.2 mg/ml. A test was conducted to confirm the effects of the addition of novel Neurospora crassa-derived proteins (TF1 and TF2) to commercially available cellulase preparations on cellulose degradation using biomass (Castanopsis sieboldii, cedar, and napier grass) as substrates.

A reaction solution was prepared to have a volume of 200 μl containing 5 μl of the protein preparation. Specifically, to observe saccharification-accelerating effects on cellulase, Trichoderma reesei cellulase and NOVO188 were mixed at a ratio of 5:1, in addition to the expression protein, so that the protein concentration was 0.2 mg/ml. Then a reaction system containing 5 μl of the solution was prepared. Therefore, 1 μg of the protein preparation was dissolved in 200 μl of the reaction system. As a substrate, Castanopsis sieboldii, cedar, napier grass, or steamed napier grass was added to a final concentration of 5% (w/v). Biomass was used after washing to cause the supernatant to lose its color under heat treatment at 60° C. for removal of soluble sugar.

Reaction was performed at 45° C. for 16 hours. An appropriate amount of a supernatant of the reaction solution was added to a tetrazolium blue dye solution (prepared by mixing 0.2% tetrazolium blue 0.1M NaOH solution with 1 M sodium potassium tartrate solution at 1:1) and then the resultant was boiled for 10 minutes for coloring. Absorbance was measured at 660 nm and thus the quantity of soluble sugar reduction ends was determined.

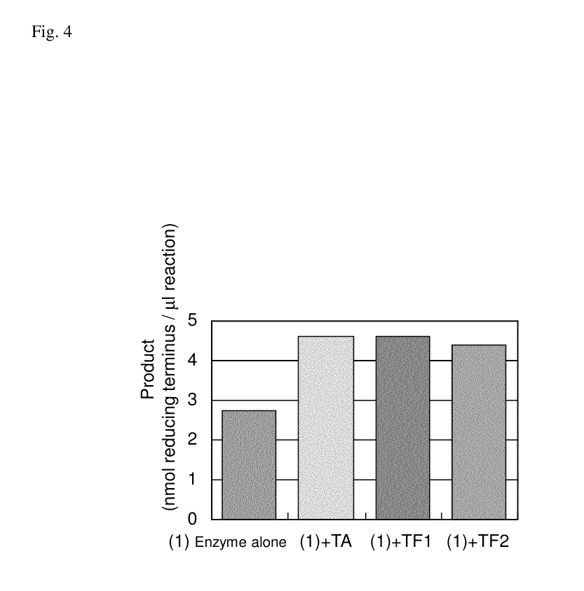

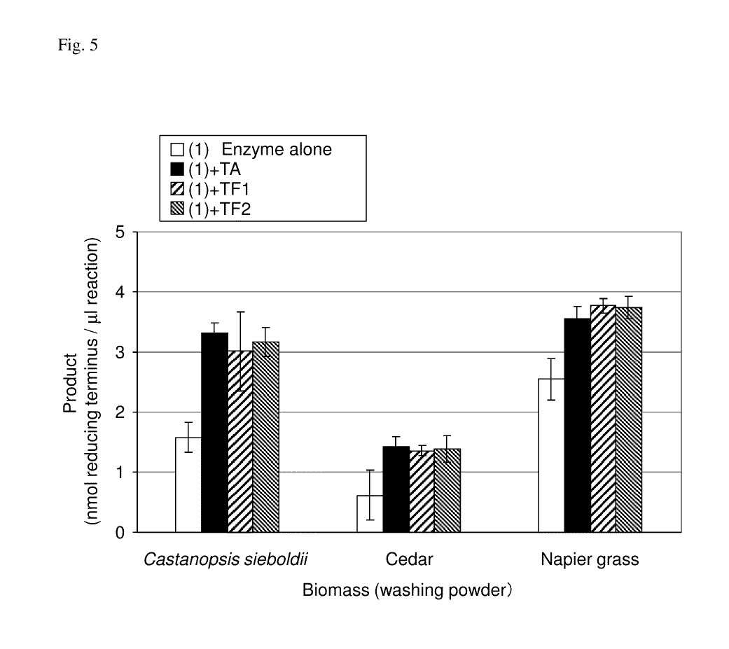

FIG. 4 shows the test results obtained when steamed napier grass was used as biomass. FIG. 5 shows the test results obtained when Castanopsis sieboldii, cedar, and napier grass were used as biomass. As shown in FIG. 4 and FIG. 5, each group to which only cellulase had been added was compared with the relevant groups to which cellulase and the expression protein had been added simultaneously. It was confirmed in all cases that when the expression protein had co-existed with cellulase, the amount of soluble sugar generated by cellulase increased about twofold. In addition, although not shown in FIG. 4 and FIG. 5, when only the expression protein had been added to biomass, no soluble sugar was detected.

It was revealed by these results that Neurospora crassa-derived GH61 (TF1) and Neurospora crassa-derived GH61 (TF2) have a function of enhancing the saccharifying activity of cellulase in a manner similar to conventionally known TA.

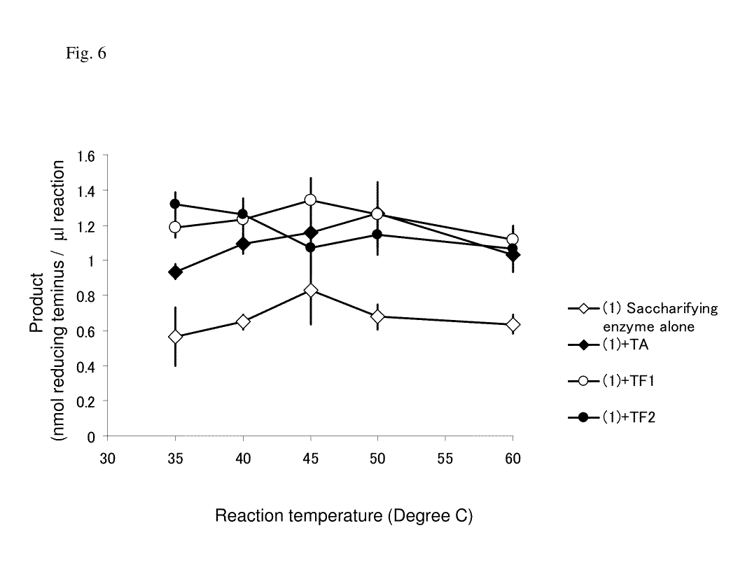

Furthermore, in this example, a test for evaluating biomass degradation was performed by a similar method except that the reaction temperature ranged from 35° C. to 60° C. FIG. 6 shows the results of plotting the amounts of degraded products at each reaction temperature. As shown in FIG. 6, it was revealed that Neurospora crassa-derived GH61 (TF1) and Neurospora crassa-derived GH61 (TF2) have a function of enhancing the saccharifying activity of cellulase within relatively low temperature ranges, unlike conventionally known TA. Specifically, Neurospora crassa-derived GH61 (TF1) was observed to exhibit a significant effect of enhancing the saccharifying activity of cellulose, particularly within a temperature region of 45° C. or less, compared with TA. Moreover, Neurospora crassa-derived GH61 (TF2) was observed to exhibit a significant effect of enhancing the saccharifying activity of cellulose, particularly within a temperature region of 40° C. or less, compared with TA.

### Example 5

### Alcohol Fermentation Using Yeast Expressing Crystalline Cellulose Binding Protein

SSCF (simultaneous saccharification and co-fermentation) was performed using genetically recombined yeast expressing the protein (obtained in Example 3 above) and pre-treated biomass (napier grass) as a substrate. A test was conducted to confirm the effects of the novel Neurospora crassa-derived protein on biomass saccharification/fermentation.

50 mM citrate buffer, a saccharifying enzyme mixture 30 FPU/g biomass (Trichoderma reesei cellulase and Novo188 mixed at a ratio of 5:1), 1% yeast extract, 2% peptone solution, and a culture solution (OD₆₀₀ₙₘ=60) of genetically recombined yeast performing 600 nm secretory expression of the above crystalline cellulose binding protein (TF1, TF2, or TA) were added to 1.0 g of pre-treated napier grass (water content: 71.5%). The solution was adjusted to a total volume of 30 ml. As a control, the S. cerevisiae YPH499 strain; that is, the host strain of the recombinant yeast, was prepared under similar conditions. As each yeast culture solution, a sample obtained after 3 days of shake culture at 30° C. and 120 rpm as preculture was used. Each of the thus prepared solution was caused to undergo simultaneous saccharification and fermentation at 30° C. and 100 rpm, and then samples were collected in a timely manner. Each of the collected samples was purified in a column, and then the ethanol concentration in the solution was measured with a biosensor (Oji Scientific Instruments).

Furthermore, as a control for the above fermentation test, a sample was also prepared by externally adding 28.5 mg of each partially purified crystalline cellulose binding protein (TF1, TF2, or TA) to the above prepared solution. The solution was caused to undergo simultaneous saccharification and fermentation at 30° C. and 100 rpm, and then samples were collected in a timely manner. Each of the collected samples was purified in a column, and then the ethanol concentration of the solution was measured using a biosensor (Oji Scientific Instruments).

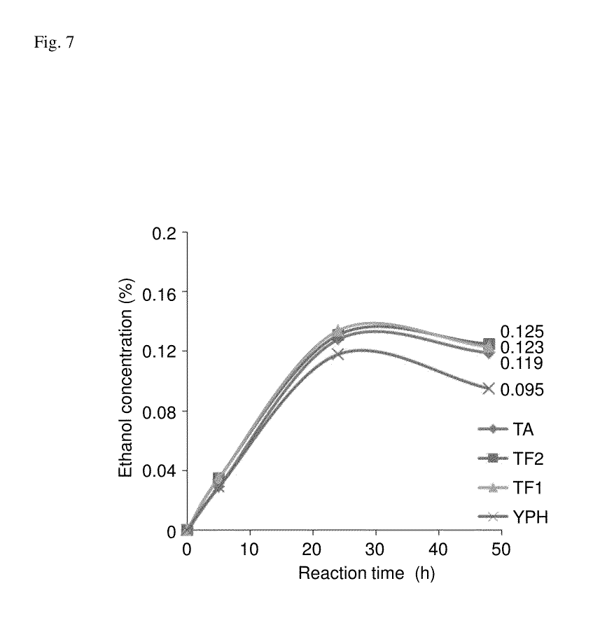

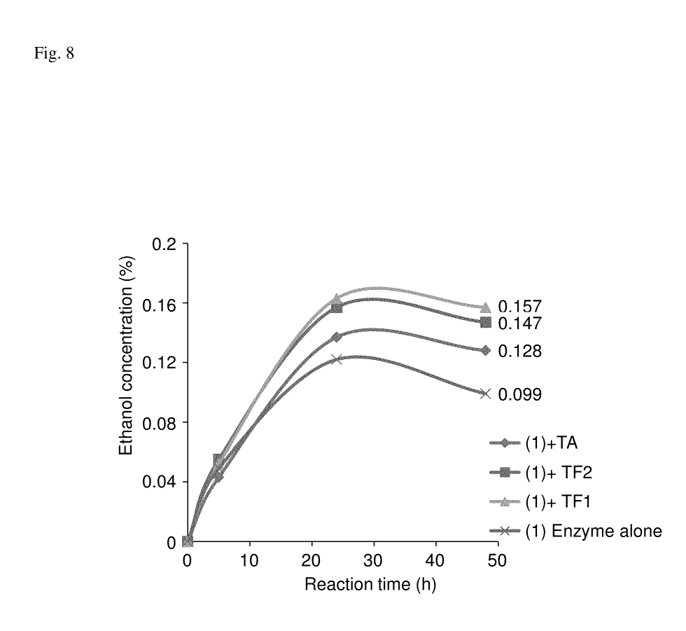

FIG. 7 shows the results of a fermentation test when each of the partially purified crystalline cellulose binding proteins was not externally added. FIG. 8 shows the results of a fermentation test when each of the partially purified crystalline cellulose binding proteins was externally added. Compared with the samples of the recombinant strain not expressing via secretory expression each crystalline cellulose binding protein or the YPH 499 strain to which no crystalline cellulose binding protein had been added, ethanol production amounts were confirmed to be higher in the samples of the strain expressing via secretory expression TF1, TF2, or TA or the sample to which the crystalline cellulose binding protein had been added. In particular, in the test in which the partially purified proteins had been externally added, higher ethanol production amounts were confirmed for TF1 and TF2 than for TA.

These examples revealed that Neurospora crassa-derived GH61 (TF1) and Neurospora crassa-derived GH61 (TF2) can improve ethanol productivity by separately existing in reaction systems for simultaneous saccharification and fermentation.

What is claimed is:

1. An isolated polypeptide according to any one of the following (a) to (c):
(a) a polypeptide comprising the amino acid sequence shown in SEQ ID NO: 2 or 4;
(b) a polypeptide having 70% or more identity with the amino acid sequence shown in SEQ ID NO: 2 or 4 and having a function of enhancing the saccharifying activity of cellulase; and
(c) a polypeptide having an amino acid sequence that has a substitution, a deletion, an addition, or an insertion of one or a plurality of amino acid residues with respect to the amino acid sequence shown in SEQ ID NO: 2 or 4, and having a function of enhancing the saccharifying activity of cellulase.

2. The polypeptide according to claim 1, which is capable of binding to crystalline cellulose.

3. The polypeptide according to claim 1, which is derived from Neurospora crassa.

4. A gene encoding the polypeptide of claim 1.

5. An expression vector containing the gene of claim 4.

6. A saccharification method, comprising a step of saccharifying cellulosic biomass with cellulase in the presence of the polypeptide of claim 1.

7. A method for producing alcohol, comprising a step of saccharifying cellulosic biomass with cellulase in the presence of the polypeptide of claim 1, and performing alcohol fermentation using a sugar component as a raw material.

8. The method for producing alcohol according to claim 7, wherein the alcohol fermentation is performed using a recombinant microorganism transformed with a gene encoding the polypeptide.

9. The method for producing alcohol according to claim 8, wherein the recombinant microorganism is recombinant yeast.

10. A composition for enhancing cellulase activity comprising the polypeptide of claim 1.
